(19) United States
(12) Patent Application Publication
(10) Pub. No.: US 2004/0191312 A1
(43) Pub. Date: Sep. 30, 2004

(54) 5-HT3 receptor agonists as neuroprotectors

(76) Inventors: Donna Oksenberg, Palo Alto, CA (US); Roman Urfer, Belmont, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 10/745,760

(22) Filed: Dec. 23, 2003

Related U.S. Application Data
(60) Provisional application No. 60/437,050, filed on Dec. 31, 2002.

(51) Int. Cl. A61K 9/48; A61K 9/20

(52) U.S. Cl. 424/464; 424/451

(57) ABSTRACT

The invention discloses methods and compositions useful for treating and preventing neurodegenerative diseases. The methods and compositions utilize agonists for the 5-HT3 receptors. These molecules can be delivered alone or in combination with agents which treat or prevent neurodegenerative diseases such as those caused by ischemic stroke, Alzheimer's disease, diabetic peripheral neuropathy, multiple sclerosis, amyotrophic lateral sclerosis, traumatic brain injury, spinal cord injury, Huntington's disease or Parkinson's disease.

### CROSS REFERENCE TO RELATED APPLICATION

This application claims priority from provisional application U.S. Serial No. 60/437,050, filed Dec. 31, 2002, the entire contents of which are incorporated by reference for all purposes.

### FIELD OF THE INVENTION

The present invention relates to the use of 5-HT3 receptor agonists to modulate neurodegenerative diseases, compounds that are 5-HT3 receptor agonists, pharmaceutical compositions containing them and methods for their use.

### BACKGROUND OF THE INVENTION

The neurotransmitter serotonin, also referred to as 5-hydroxytryptamine (5-HT), was first discovered in 1948 and subsequently has been the subject of an immense quantity of research. Serotonin acts both centrally and peripherally on discrete 5-HT receptors. The 5-HT receptors have been delineated into three major sub-classifications—5-HT1, 5-HT2 and 5-HT3, and at least fourteen subtypes of serotonin receptors have been pharmacologically defined. Thirteen of the fourteen known receptors are G-protein coupled receptors, and the only known ionotropic 5-HT receptor, the type 3,5-HT3 receptor, is a fast activating, ligand-gated non-selective cation channel—unique among known monoamine receptors. The 5-HT1 and 5-HT2 receptors are described in U.S. Pat. No. 5,155,218, and they are characterized by seven transmembrane regions.

The 5-HT3 receptors are ligand-gated ion channels that are related structurally to the GABA, glycine and nicotinic acetylcholine receptors. Receptors of the 5-HT3 subclass pervade autonomic neurons and appear to regulate the release of a variety of neurotransmitters in the gastrointestinal, cardiovascular and central nervous systems. Activation of the 5-HT3 receptor leads to membrane depolarization. Activation of the 5-HT3 receptors mediate transient inward currents and membrane depolarization under physiological conditions. In the CNS the functional properties of presynaptic 5-HT3 receptors may differ from those of postsynaptic 5-HT3 receptors.

The presynaptic receptors are calcium permeable ion channels and the influx of calcium through these channels is responsible for the elevation of intracellular calcium induced by stimulation with 5-HT. Conversely postsynaptic 5-HT receptors on hippocampal interneurons are blocked by calcium at negative membrane potentials. There are two known subunits for the human 5-HT3 receptor: 5-HT3R-A and 5-HT3R-B. They have structural and functional similarities with nicotinic, GABA-ergic and other ligand gated ion channels. The 5-HT3R-A subunit is a protein with four transmembrane segments and an extracellular N-terminus, whereas 5-HT3R-B subunit is more complex. Its transmembrane segments are not homologous to other ligand-gated ion channels (Maricq et al. (1991) Science 254: 432-437 and Davies et al. (1999) Nature 397: 359-363). Apparently the extracellular N-terminal domain of the 5-HT3R-A subunit contains the agonist recognition site (Spier et al. (2000) J. Biol. Chem. 275: 5620-5626).

The 5-HT3 receptor is the target of antagonists (granisetron and ondansetron) selective against the nausea induced by cytotoxic chemotherapy and general anesthesia. There is some evidence that serotonin 5-HT3 receptors are important in pain reception, anxiety, cognition, cranial motor neuron activity, sensory processing, modulation of affect, and the behavioral consequences of drug. The known 5-HT3 agonists include 5-HT, 2-methyl-5-HT, 1-phenylbiguanide and m-chlorophenylbiguanide with an EC₅₀ of 2.0, 5.0-8.0, 22.0, and 0.4 μM, respectively (van Hooft et al. (1997) J. Neurochem. 69: 1318-132.1, and Morain et al. (1994) Mol. Pharmacol. 46: 732-742), and SR 57227A (1-(6-Chloro-2-pyridinyl-4-piperidinamine hydrochloride), quipazine N-methyl-dimaleate (2-[1-(4-methyl)-piperazinyl]quinoline dimaleate, and YM-31636, a novel 5-HT3 agonist described by Ito et al. (Eur J Pharmacol 409 195-201, 2000). In addition, EP-0,749,966 discloses fused thiazole derivatives as 5-HT3 agonists useful for the treatment of gastrointestinal tract diseases, EP-0,666,263 discloses condensed thiazole derivates as 5-HT3 agonists useful for the treatment of gastrointestinal disorders and migraines, and EP-0,506,545 discloses the use of 4-amino-1-(2-pyridyl)piperidines as 5-HT3 agonists for the treatment of serotoninergic dysfunctions.

The 5-HT3 receptors are exclusively localized on neurons in the central and peripheral nervous systems. Therefore, agonists for the receptor could be used for the treatment and prevention of neurodegenerative diseases. Neurodegenerative diseases are characterized by the dysfunction and death of neurons, leading to the loss of neurologic functions mediated by the brain, spinal cord and the peripheral nervous system. These disorders have a major impact on society. For example, approximately 4 to 5 million Americans are afflicted with the chronic neurodegenerative disease known as Alzheimer's disease. Other examples of chronic neurodegenerative diseases include diabetic peripheral neuropathy, multiple sclerosis, amyotrophic lateral sclerosis, traumatic brain injury, spinal cord injury, Huntington's disease and Parkinson's disease. Normal brain aging is also associated with loss of normal neuronal function and may entail the depletion of certain neurons.

Though the mechanisms responsible for the dysfunction and death of neurons in neurodegenerative disorders are not well understood, a common theme is that loss of neurons results in both the loss of normal functions and the onset of adverse behavioral symptoms. Therapeutic agents that have been developed to retard loss of neuronal activity and survival have been largely ineffective. Some have toxic side effects that limit their usefulness. Other promising therapies, such as neurotrophic factors, are prevented from reaching their target site because of their inability to cross the blood-brain barrier. To date rTPA (recombinant tissue plasminogen activator), a thrombolytic serine protease, is the only therapeutic specifically indicated to treat ischemic stroke in the United States and Canada. In order to be treated with rTPA the patient has to be treated within 3 hours after the onset of symptoms and it is believed that only 3-5% of patients fall in this category. TPA is contra-indicated for a variety of conditions that will increase the risk of bleeding. So far no agent that acts at the neurobiochemical level has shown clinical efficacy in stroke therapy.

Stroke is the third ranking cause of death in the United States, and accounts for half of neurology inpatients. Depending on the area of the brain that is damaged, a stroke can cause coma, paralysis, speech problems and dementia. The five major causes of cerebral infarction are vascular thrombosis, cerebral embolism, hypotension, hypertensive hemorrhage, and anoxia/hypoxia.

The brain requires glucose and oxygen to maintain neuronal metabolism and function. Hypoxia refers to inadequate delivery of oxygen to the brain, and ischemia results from insufficient cerebral blood flow. The consequences of cerebral ischemia depend on the degree and duration of reduced cerebral blood flow. Neurons can tolerate ischemia for 30-60 minutes, but reperfusion must be reestablished before 3-6 hours of ischemia have elapsed. Neuronal damage can be less severe and reversible if flow is restored within a few hours, providing a window of opportunity for intervention. Thus, a need exists for therapies that can protect the brain during a stroke, that can prevent reperfusion injury after a stroke and that can restore the function of brain cells. The present invention fulfills these and other needs.

### SUMMARY OF THE INVENTION

The present invention provides methods and compositions for treating and preventing neurodegenerative diseases. The present invention is based on the discovery that 5-HT3 agonists exhibit neuroprotective effect. These molecules can be delivered alone or in combination with additional agents, and are used for the treatment and/or prevention of neurodegenerative diseases such as those resulting from ischemic strokes.

Accordingly, in one aspect, the subject invention is directed to a method for treating or preventing neurodegenerative disease in a subject in need thereof. The method comprises administering to the subject a pharmaceutically effective amount of an agonist for the 5-HT3 receptor.

These and other aspects of the present invention will become evident upon reference to the following detailed description. In addition, various references are set forth herein which describe in more detail certain procedures or compositions, and are therefore incorporated by reference in their entirety.

The invention thus provides methods for treating or preventing neurodegenerative disease in a mammalian subject in need thereof, the method comprising administering a pharmaceutically effective amount of an agonist for the 5-HT3 receptor to the subject. The neurodegenerative disease can be ischemic stroke, Alzheimer's disease, diabetic peripheral neuropathy, multiple sclerosis, amyotrophic lateral sclerosis, traumatic brain injury, spinal cord injury, Huntington's disease or Parkinson's disease, but is preferably ischemic stroke, traumatic brain injury, or Alzheimer's disease. Further, the invention provides methods for administering an additional active agent. The agonists of the invention are administered in a pharmaceutical composition containing a pharmaceutically acceptable excipient. The excipient is suitable for oral administration. Thus, the composition is in the form of a tablet, a capsule, or a soft-gel capsule. In addition, the excipient is liquid suited to intravenous, intramuscular, or subcutaneous administration. Further, the excipient is suited to transdermal administration, or buccal administration. The 5-HT3 receptor can be a 5-HT3R-A receptor, a 5-HT3R-B receptor, or a recombinant 5-HT3 receptor. The 5-HT3 agonist can be 1-(m-chlorophenyl)-biguanide, N-phenyl-imidocarbonimidicdiamide, 2-methyl-5-hydroxytryptaminemaleate, 2-(1-(4-methyl)-piperazineyl)quinolinedimaleate, 1-(6-chloro-2-pyridinyl)-4-piperidinamine, or salts, or solvates thereof.

In another aspect, the invention provides a 5-HT3 agonist that is 1 -(m-chlorophenyl)-biguanide, N-phenyl-imidocarbonimidicdiamide, 2-methyl-5-hydroxytryptaminemaleate, 2-(1-(4-methyl)-piperazineyl)quinolinedimaleate, 1-(6-chloro-2-pyridinyl)-4-piperidinamine, or salts, or solvates thereof. The invention further provides pharmaceutical compositions comprising a therapeutically effective amount of at least one 5-HT3 agonists in admixture with at least one pharmaceutically acceptable carrier. The pharmaceutical compositions can be used in a method of neuroprotecting a patient comprising administering to a patient a therapeutically effective amount of at least 5-HT3 agonists or its pharmaceutical composition. The compositions and methods can be used to treat a patient suffering from a disease or prevent a patient from contacting a disease such as ischemic stroke, Alzheimer's disease, diabetic peripheral neuropathy, multiple sclerosis, amyotrophic lateral sclerosis, traumatic brain injury, spinal cord injury, Huntington's disease, Parkinson's disease, and the like.

### DETAILED DESCRIPTION

I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry ₃rd Ed.” Vols. A and B, Plenum Press, New York. The practice of the present invention will employ, unless otherwise indicated, conventional methods of mass spectroscopy, protein chemistry, biochemistry, recombinant DNA techniques and pharmacology, within the skill of the art.

The term “modulator” means a molecule that interacts with a target. The interactions include, but are not limited to, agonist, antagonist, and the like, as defined herein.

The term “agonist” means a molecule such as a compound, a drug, an enzyme activator or a hormone that enhances the activity of another molecule or the activity of the 5-HT3 receptor site.

The term “antagonist” means a molecule such as a compound, a drug, an enzyme inhibitor, or a hormone, that diminishes or prevents the action of another molecule or the activity of the 5-HT3 receptor site.

The term “stroke” broadly refers to the development of neurological deficits associated with impaired blood flow to the brain regardless of cause. Potential causes include, but are not limited to, thrombosis, hemorrhage and embolism. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. The term “ischemic stroke” includes cerebral ischemia after cardiac arrest, stroke, and multi-infarct dementia, including those resulting from surgery. Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

By “neuroprotective agent” as used herein is meant a compound effective to reduce neuronal cell death, including the ability to inhibit the spread of neuronal damage from the initial site of injury. 5-HT3 agonist compounds of the present invention selected by the in vitro screening methods are thus predicted to be neuronal-cell specific, neuroprotective agents.

The term “alkyl” means the monovalent branched or unbranched saturated hydrocarbon radical, consisting solely of carbon and hydrogen atoms, having from one to twelve carbon atoms inclusive, unless otherwise indicated. Examples of alkyl radicals include, but are not limited to, methyl, ethyl, propyl, isopropyl, butyl, isobutyl, sec-butyl, tert-butyl, pentyl, n-hexyl, octyl, dodecyl, and the like.

The term “alkylene” as used herein means the divalent linear or branched saturated hydrocarbon radical, consisting solely of carbon and hydrogen atoms, having from one to eight carbon atoms inclusive, unless otherwise indicated. Examples of alkylene radicals include, but are not limited to, methylene, ethylene, trimethylene, propylene, tetramethylene, pentamethylene, ethylethylene, and the like.

The term “alkenylene” means the divalent linear or branched unsaturated hydrocarbon radical, containing at least one double bond and having from two to eight carbon atoms inclusive, unless otherwise indicated. The alkenylene radical includes the cis or trans ((E) or (Z)) isomeric groups or mixtures thereof generated by the asymmetric carbons. Examples of alkenylene radicals include, but are not limited to ethenylene, 2-propenylene, 1-propenylene, 2-butenyl, 2-pentenylene, and the like.

The term “aryl” means the monovalent monocyclic aromatic hydrocarbon radical consisting of one or more fused rings in which at least one ring is aromatic in nature, which can optionally be substituted with hydroxy, cyano, lower alkyl, lower alkoxy, thioalkyl, halogen, haloalkyl, hydroxyalkyl, nitro, alkoxycarbonyl, amino, alkylamino, dialkylamino, aminocarbonyl, carbonylamino, aminosulfonyl, sulfonylamino, and/or trifluoromethyl, unless otherwise indicated. Examples of aryl radicals include, but are not limited to, phenyl, naphthyl, biphenyl, indanyl, anthraquinolyl, and the like.

The term “halogen” as used herein refers to fluoro, bromo, chloro and/or iodo.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a 5-HT3 agonist disclosed herein required to provide a clinically significant decrease in neurodegenerative disease, such as those resulting from ischemic stroke. An appropriate “effective” amount in any individual case may be determined by one of ordinary skill in the art using routine experimentation.

As used herein, the terms “treat” or “treatment” are used interchangeably and are meant to indicate a postponement of development of neurodegenerative diseases and/or a reduction in the severity of such symptoms that will or are expected to develop. The terms further include ameliorating existing neurodegenerative symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The term “pharmaceutically acceptable salt” of a compound means a salt that is pharmaceutically acceptable and that possesses the desired pharmacological activity of the parent compound. Such salts, for example, include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;

(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The compounds of the present invention may be used as agonists for the 5-HT3 receptors. In this context, the increase in the channel activity refers to a higher level of measured activity relative to a control experiment in which the channel, cell, or subject is not treated with the test compound. In particular embodiments, the agonist results in at least a 10% increase. One of skill in the art will appreciate that the increase of the measured activity of at least 20%, 50%, 75%, 90% or 100% or any integer between 10% and 100%, may be preferred for particular applications.

II. Method of Identifying the Targets

In accordance with the present invention, compounds that are 5-HT3 agonists are useful for preventing and treating disease conditions associated with ischemic cell death, such as myocardial infarction, stroke, glaucoma, and other neurodegenerative diseases. Various neurodegenerative diseases which may involve apoptotic cell death, include, but are not limited to, Alzheimer's Disease, ALS and motor neuron degeneration, Parkinson's disease, peripheral neuropathies, Down's Syndrome, age related macular degeneration (ARMD), traumatic brain injury, spinal cord injury, Huntington's Disease, spinal muscular atrophy, and HIV encephalitis. The 5-HT3 agonists can be used in methods and compositions for imparting neuroprotection and for treating neurodegenerative diseases.

Several 5-HT3 agonists are known which may find use with the subject methods. In addition, other equivalent agents may also be used as agonists to increase the activity of the 5-HT3 receptors in subjects.

III. Methods for identifying agents for treating or preventing neurodegenerative diseases

Several methods for identifying agonists for the 5-HT3 receptors that may also treat and/or prevent neurodegenerative diseases may be employed. One method used to identify compounds that are 5-HT3 agonists involves placing cells, tissues, or preferably a cellular extract or other preparation containing 5-HT3 receptors in contact with several known concentrations of a test compound in a buffer compatible with 5-HT3 receptors activity. Other methods for determining the agonist concentration of a compound of the invention against 5-HT3 receptors can be employed as will be apparent to those of skill in the art based on the disclosure herein.

The cell cultures are used in screening agents for their effect on neural and/or brain cells and neurologic events, e.g. during ischemia. Such agents may include candidate drug compounds, genetic agents, e.g. coding sequences; polypeptides, e.g. factors, antibodies, etc.; and physiologic conditions, e.g. glucose, oxygen, etc.

Oxygen and glucose deprivation (OGD) in cell cultures has been studied by exposing cultured tissue to media such as artificial cerebro-spinal fluid (aCSF), with an ion composition similar to that of the extracellular fluid of normal brain, with 2-6 mM K⁺, 1.5-3 mM Ca²⁺, 116 mM NaCl, 1 mM NaH₂PO₄, 26.2 mM NaHCO3, 0.01 mM glycine in a glucose free media, and pH 7.4. The cells are maintained in the ischemic conditions for a period of time sufficient to induce a detectable effect, usually for at least about 90 min, preferably for at least about 60 minutes, and for not more than about 2 hours.

However, during ischemia the distribution of ions across cell membranes dramatically shift. The copending application provides a medium that more accurately reflects the extracellular fluid of the brain during an ischemic event. In another embodiment of a method for identifying agonists, described in the copending and coowned application, U.S. Ser. No. 10/131,731, the conditions and culture medium allow simulation of physiological and pathophysiological events affecting neural cells. Cultures of suitable cells or hippocampal slices are exposed transiently to a synthetic medium that reproduces the effects of ischemia. The cells or the slices are then monitored for the effect of the ischemic conditions on physiology, phenotype, etc.

In one aspect, the cells are an integrated system of brain tissue, with preserved synaptic connections and a diversity of cells including neurons, astrocytes and microglia. Such tissue can provide an in vitro model for pathophysiological events in the hippocampus following ischemia in vivo, including selective and delayed neuronal death in the CA1 region and increased damage by hyperglycemia.

Artificial ischemic cerebro-spinal fluid (iCSF), as used herein, refers to a glucose-free medium similar to the extracellular fluid of the brain during ischemia in vivo. The iCSF ionicity has a potassium concentration of at least about 50 mM, not more than about 90 mM, usually at least about 60 mM, not more than about 80 mM, and preferably about 65 to 75 mM K⁺, and in some instances about 70 mM K⁺. The concentration of calcium is at least about 0.1 mM, not more than about 1 mM, usually at least about 0.2 mM and not more than about 0.5 mM, preferably about 0.3 mM Ca²⁺. The pH of the iCSF media is at least about 6.7 and not more than about 6.9, preferably about pH 6.8.

The medium may be glucose free, or may comprise glucose at a concentration from about 10 mM to 100 mM, usually from about 25 mM to 75 mM, and may be about 40 mM. The cultures of the present invention show increased cell damage in the presence of glucose during ischemia, which simulates the in vivo effects of glucose. Hyperglycemia aggravates ischemic brain damage in vivo, and glucose in iCSF also significantly exacerbates cell damage following oxygen deprivation. This model of in vitro ischemia is useful in studies of the mechanisms and treatment of ischemic cell death.

The cells or hippocampal slices are maintained in the ischemic conditions for a period of time sufficient to induce a detectable effect, usually for at least about 5 minutes, more usually for at least about 1 minute, preferably for at least about 15 minutes, and for not more than about 1 hour.

Maintaining cultured cells or hippocampal slices in vitro in iCSF during oxygen glucose deprivation (OGD) provides a realistic simulation of in vivo events, which include a selective and delayed cell death in the CA1 region, assessed by propidium iodide uptake. Cell death is glutamate receptor dependent, as evidenced by the mitigation of damage by blockade of the N-methyl-D-aspartate and the α-Amino-3-hydroxy-5-methyl-4-isoxazolepropionic acid receptors.

Screening methods generally involve conducting various types of assays to identify agents that affect tissue damage that occurs during ischemia. Thus, a commercially available library of compounds is screened for potential neuroprotective compounds against oxygen-glucose deprivation (OGD) induced cell death in neuronal primary cultures. In brief when neurons are deprived of chemical energy, glutamate floods out of the neurons in which it is stored and over activates receptors in nearby cells. These receptors are ionotropic ion channels leading to entry of deadly amount of calcium and sodium into the cells and causing a delayed cell death after 24 hours in culture. These conditions mimic the ischemic stroke. The OGD induced cell death screening method was used to investigate ability of the commercially available Sigma Library of Pharmacologically Active Compounds (LOPAC) to identify compounds that protect against OGD-induced cell death. The following compounds were identified as 5-HT3 agonists:

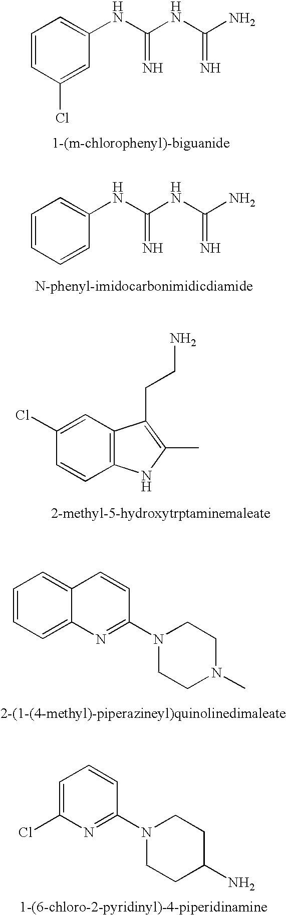

The 5-HT3 agonists I-V were identified as being neuroprotective against OGD-induced cell death.

In another method of Nelson and Thomas (1989) Biochemical Pharmacology 38: 1693-1695, a 5-HT₃ receptor binding test can be run using, as a tracer ligand, endo-1-methyl-N-(9-methyl-9-azabicyclo[3.3.1 ]non-3-yl)-1H-indazole-3-carboxamide substituted by tritium ([³H]granisetron). Crude synapse membrane sample can be prepared from rat cerebral cortex, suspended in 50 mM HEPES buffer (pH 7.4) and used for testing. Several different concentrations of the test compounds and [³H]granisetron (final concentration 1.0 nM) are usually added to this suspension and allowed to react at 25° C. Thirty minutes later, the reaction mixture can be filtered by suction with cell harvester. The filter can be washed with a buffer, such as pH 7.4 Tris buffer, and the radioactivity on the filter can be counted using liquid scintillation counter or Microbeta plate.

In another method, rational drug design, based upon structural studies of the molecular shapes of the 5-HT3 agonists identified above, serotonin, other effectors or analogs, or the receptors, may be used to identify compounds whose three-dimensional structure is complementary to that of the active site of the 5-HT3 receptors. These compounds may be determined by a variety of techniques, including molecular mechanics calculations, molecular dynamics calculations, constrained molecular dynamics calculations in which the constraints are determined by NMR spectroscopy, distance geometry in which the distance matrix is partially determined by NMR spectroscopy, x-ray diffraction, or neutron diffraction techniques. In the case of all these techniques, the structure can be determined in the presence or absence of any ligands known to interact with 5-HT3 receptors.

Such computer programs include but are not limited to AMBER (available from University of California, San Francisco), CHARMM (Chemistry at HARvard Molecular Mechanics, available from Harvard University), MM2, SYBYL (Trypos Inc.), CHEMX (Chemical Design), MACROMODEL, GRID (Molecular Discovery Ltd), and Insight II (Accelry). Such programs are contemplated as being useful for the determination of the chemical interaction between two molecules, either isolated, or surrounded by solvent molecules, such as water molecules, or using calculations that approximate the effect of solvating the interacting molecules. The relative orientation of the two can be determined manually, by visual inspection, or by using other computer programs which generate a large number of possible orientations. Examples of computer programs include but are not limited to DOCK and AutoDOCK. Each orientation can be tested for its degree of complementarity using the computer programs. Thus, novel compounds can be designed that are capable of acting as 5-HT3 agonists.

Other methods for identifying compounds that may increase the activity of the 5-HT3 receptors involve the use of techniques such as UV/VIS spectroscopy, polarimetry, CD or ORD spectroscopy, IR or Raman spectroscopy, NMR spectroscopy, fluorescence spectroscopy, HPLC, gel electrophoresis, capillary gel electrophoresis, dialysis, refractometry, conductometry, atomic force microscopy, polarography, dielectometry, calorimetry, solubility, EPR, surface plasmon resonance, or mass spectroscopy. The application of these methods can be direct, in which the compound's interaction with 5-HT3 receptors is measured directly, or it can be indirect, in which a particular agent having a useful spectroscopic property is used as a probe for the ability of other compounds to interact with the 5-HT3 receptors; for example, by displacement or by fluorescence quenching.

The 5-HT3 agonists thus identified or designed can be subsequently tested for their ability to treat and/or prevent neurodegenerative diseases. In one embodiment, the computer based methods discussed above are used. In another method, the compounds are tested for their ability to modulate 5-HT3 receptors, such as, for example, 5-HT3R-A, 5-HT3R-B, or recombinant 5HT3. Lead compounds identified during these screens can serve as the basis for the synthesis of more active analogs. Lead compounds and/or active analogs generated therefrom can be formulated into pharmaceutical compositions effective in treating neurological disorders such as stroke, epilepsy and neurodegenerative disorders. The compositions and methods of the present invention thus provide a means of imparting neuroprotection to a subject.

IV. Synthesis of the 5-HT3 Agonists

The compounds of the invention comprise the 5-HT3 agonists, as described above. The 5-HT3 agonists compounds of the present invention, and other related compounds having different substituents identified by any of the methods described above can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 3^{rd} Ed., Vols. A and B (Plenum 1992), and Green and Wuts, PROTECTIVE GROUPS IN ORGANIC SYNTHESIS 2^{nd} Ed. (Wiley 1991). Starting materials for the compounds of the invention may be obtained using standard techniques and commercially available precursor materials, such as those available from Aldrich Chemical Co. (Milwaukee, Wis.), Sigma Chemical Co. (St. Louis, Mo.), Lancaster Synthesis (Windham, N.H.), Apin Chemicals, Ltd. (New Brunswick, N.J.), Ryan Scientific (Columbia, S.C.), Maybridge (Cornwall, England) and Trans World Chemicals (Rockville, Md.).

The procedures described herein for synthesizing the compounds of the invention may include one or more steps of protection and deprotection (e.g., the formation and removal of acetal groups). In addition, the synthetic procedures disclosed below can include various purifications, such as colunm chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. Methods of protection and deprotection, purification and identification and quantification are well known in the chemical arts.

V. Pharmaceutical Formulations and Modes of Administration

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, the compounds of this invention will be administered in a therapeutically effective amount by any of the accepted modes of administration. Suitable dosage ranges are 1-2500 mg daily, preferably 1-1500 mg daily, and most preferably 1-500 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of this invention for a given disease.

In general, compounds of this invention will be administered as pharmaceutical formulations including those suitable for oral (including buccal and sub-lingual), rectal, nasal, topical, pulmonary, vaginal or parenteral (including intramuscular, intraarterial, intrathecal, subcutaneous and intravenous) administration or in a form suitable for administration by inhalation or insufflation. The preferred manner of administration is intravenous using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

For oral administration, the composition will generally take the form of a tablet, capsule, a softgel capsule or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils, fatty esters or polyols are conventionally employed as solvents or suspending media. In addition, parenteral administration may involve the use of a slow release or sustained release system such that a constant level of dosage is maintained.

Alternatively, the pharmaceutical compositions of the invention may be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the invention may also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or gel reservoir, or may take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount will be in the range of about 0.05 mg/kg to about 40 mg/kg body weight, more preferably about 0.5 mg/kg to about 20 mg/kg, in at least one dose. In larger mammals the indicated daily dosage can be from about 1 mg to 100 mg, one or more times per day, more preferably in the range of about 10 mg to 50 mg. The subject may be administered as many doses as is required to reduce and/or alleviate the signs, symptoms, or causes of the disorder in question, or bring about any other desired alteration of a biological system.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

As discussed above, the pharmaceutical formulations may contain one or more 5-HT3 agonists and additionally one or more active agents that effectively provide neuroprotection to the subject. The additional active agent may be, but is not limited to, a 5-HT3 antagonist, a GABA antagonist or an agonist, a NSAID, 5-HT1A ligand, sigma receptor ligand, a COX-2 inhibitor, or another pain killer, a vitamin, or a hormone, and combinations thereof. This additional active agent can be administered to the subject prior to, concurrently with or subsequently to administration of the 5-HT3 agonists of this invention.

VI. Kits

In another aspect, the invention relates to pharmaceutical compositions in kit form. The kit comprises container means for containing the compositions such as a bottle, a foil packet, or another type of container. Typically the kit further comprises directions for the administration of the compositions. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be administered. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent, such as, for example, a mechanical counter which indicates the number of daily doses that has been dispensed, a microchip memory coupled with a liquid crystal readout, or audible reminder signal which, for example, reads out the date that the last daily dose has been taken and/or reminds one when the next dose is to be taken, and the like.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

Cell Cultures

Primary neuron cultures were prepared as follows: Brain cortices from E18 rat brains were dissected in DPBS without calcium and magnesium. They were digested at 37° C. for 30 min with trypsin. The digestion was stopped by addition of MEM, 10% Fetal Bovine Serum (FBS)and 10% Horse Serum. DNase (10 U/ml) was also added. The tissues were mechanically triturated using a pipette. This step was repeated until all cells were dissociated and then they were filtered through a 70 um Nitrex mesh. The cells were counted and plated in poly ornithine-coated 96-well culture plates at 5×10⁵ cells/well in MEM containing 10% HS and 10% FBS. Three days later, the medium was changed to neurobasal-serum free containing B27 and SATO (Sodium selenite 4 μg/ml, transferrrin 100 μg/ml, BSA 100 μg/ml, progesterone 62 μg/ml, putrescine 160 μg/ml). 5-Fluoro-2′deoxyuridine (FdU 30 μM) was added to inhibit the dividing microglia from the cultures. The culture medium was changed every 3 days leaving half volume of the old (conditioned) media and adding half volume of fresh media. All experiments were performed in 11-12 days-old neuronal cultures. The cells were maintained at 37° C. in a 5% CO₂ humidified incubator.

### Example 2

LOPAC Library Screen Using the Oxygen-Glucose Deprivation (OGD) Assay.

The compounds from the LOPAC library, obtained from Sigma, were plated in 96-wells at 200 μM (20× the final concentration). In addition, compound V, purchased from Tocris, was included in the screening method.

The neuronal cultures were pre-incubated with the compounds (10 μM final concentration) for 2 hours. The cells were then subjected to oxygen glucose deprivation (OGD) for 120 min at 37° C. in presence of the compounds of the library. Cultures were placed in an anaerobic chamber (Forma Scientific) and washed two times with balanced salt solution (116 mM NaCl, 5.4 mM KCl, 1 mM NaH₂PO4, 1.8 mm CaCl₂, 26.2 mM NaHCO₃, 0.01 mM glycine, pH=7.4) lacking glucose and aerated with an anaerobic gas mix (85% N2/5% CO2/10% H2) to remove residual oxygen. Control cultures were kept in the original neurobasal media but were submitted to the anaerobic conditions. MK-801 (10 μM), a non competitive NMDA receptor antagonist was used to prevent cell death after OGD caused by activation of the NMDA receptors. To terminate the oxygen glucose deprivation, the cells were removed from the anaerobic chamber and the OGD media was removed and replaced with neurobasal media. The compounds were added back for an additional 20 hours.

Cells were lysed and ATP content was measured as an index for cell viability using a commercially available reagent (Cell Titter Glo from Promega). This assay generates a luminescent signal produced by the luciferase reaction. The results for compounds of Formula I-V are provided in Table 1.

TABLE 1

| | | Activity |
|---|---|---|
| Formula | Name | (% Cells Alive) |
| I | 1-(m-chlorophenyl)-biguanide | 106 ± 6 |
| II | N-phenyl-imidocarbonimidicdiamide | 67 ± 25 |
| III | 2-methyl-5-hydroxytryptaminemaleate | 106 ± 25 |
| IV | 2-(1-(4-methyl)-piperazineyl) | 103 ± 30 |
| | quinolinedimaleate | |
| V | 1-(6-chloro-2-pyridinyl)-4-piperidinamine | 80 ± 27 |

The 5-HT3 receptor agonists, compounds I-V, were identified as being neuroprotective against the OGD-induced cell death.

### Example 3

Animal Studies

Animals are given 10 mg/kg (can be between 1-20 mg/kg) of compound I, II, III, IV, or V intraperitoneally for 30 min. They are then subjected to 2 hours of transient focal ischemia by MCAO (middle cerebral artery occlusion) with an intraluminal filament technique (Toung et al. (1999) Stroke 30: 1279-1285). At the end of the ischemic procedure, they are subjected to a continuous infusion of compound for 24 hours. Alternatively, the animals are subjected to 90 minutes of transient focal ischemia by MCAO. After reperfusion animals are treated with a bolus dose(1-20 mg/kg) of compound I, II, III, IV, V or vehicle at 5 min of reperfusion in addition to a continous infusion. More extended window times for compound administration are explored. After 24 hours of reperfusion, the animals are sacrificed. The brains are harvested and sliced into coronal sections for staining with 1% triphenyltetrazolium chloride (TTC) in saline at 37° C. for 30 minutes. Infarction volume is measured by digital imaging and image analysis software. Infarction volumes are determined in cortex and striatum an expressed as a percentage of the volume of the ipsilateral structure. The animal treated with one of the compounds showed little or no cell death. The compositions thus provide neuroprotection to the animals

### Example 4

Preparation of Tablets

The compound of formula IV (10.0 g) is mixed with lactose (85.5 g), hydroxypropyl cellulose HPC-SL (2.0 g), hydroxypropyl cellulose L-HPC, LH-22 (2.0 g) and purified water (9.0 g), the resulting mixture is subjected to granulation, drying and grading, and the thus obtained granules are mixed with magnesium stearate (0.5 g) and subjected to tablet making, thereby obtaining tablets containing 10 mg per tablet of the compound of formula IV.

### Example 5

Administering to a Subject

The tablet prepared in Example 4 is provided to a subject at time 0, and one tablet every 24 h for a period of one week. After administration of the third tablet, the subject is exposed to a neurodegenerative event. The treated subject exhibits symptoms of neurological disorder that are less severe compared to the subject that was not treated.

All printed patents and publications referred to in this application are hereby incorporated herein in their entirety by this reference.

While the preferred embodiment of the invention has been illustrated and described, it will be appreciated that various changes can be made therein without departing from the spirit and scope of the invention.

We claim:

1. A method for treating or preventing neurodegenerative disease in a mammalian subject in need thereof, the method comprising administering a pharmaceutically effective amount of an agonist for the 5-HT3 receptor to the subject.

2. The method of claim 1, wherein the neurodegenerative disease is selected from the group consisting of ischemic stroke, Alzheimer's disease, diabetic peripheral neuropathy, multiple sclerosis, amyotrophic lateral sclerosis, Huntington's disease and Parkinson's disease.

3. The method of claim 2, wherein the neurodegenrative disease is ischemic stroke.

4. The method of claim 2, wherein the neurodegenrative disease is Alzheimer's disease.

5. The method of claim 1, further comprising administering an additional active agent.

6. The method of claim 1, wherein the agonist is administered in a pharmaceutical composition containing a pharmaceutically acceptable excipient.

7. The method of claim 6, wherein the excipient is suitable for oral administration.

8. The method of claim 7, wherein the excipient is a solid.

9. The method of claim 8, wherein the composition is in the form of a tablet, a capsule, or a soft-gel capsule.

10. The method of claim 6, wherein the excipient is liquid.

11. The method of claim 10, wherein the excipient is suited to intravenous, intramuscular, or subcutaneous administration.

12. The method of claim 6, wherein the excipient is suited to transdermal administration.

13. The method of claim 6, wherein the excipient is suited to buccal administration.

14. The method of claim 1, wherein the 5-HT3 receptor is a 5-HT3R-A receptor.

15. The method of claim 1, wherein the 5-HT3 receptor is a 5-HT3R-B receptor.

16. A 5-HT3 agonist, wherein the agonist is a compound selected from the group consisting of 1-(m-chlorophenyl)-biguanide, N-phenyl-imidocarbonimidicdiamide, 2-methyl-5-hydroxytryptaminemaleate, 2-(1-(4-methyl)-piperazineyl)quinolinedimaleate, and 1-(6-chloro-2-pyridinyl)-4-piperidinamine, or salts, or solvates thereof.

17. The agonist of claim 16, wherein the compound is 1-(m-chlorophenyl)-biguanide, or salts, or solvates thereof.

18. The agonist of claim 16, wherein the compound is N-phenyl-imidocarbonimidicdiamide, or salts, or solvates thereof.

19. The agonist of claim 16, wherein the compound is 2-methyl-5-hydroxytryptaminemaleate, or salts, or solvates thereof.

20. The agonist of claim 16, wherein the compound is 2-(1-(4-methyl)-piperazineyl)quinolinedimaleate, or salts, or solvates thereof.

21. The agonist of claim 16, wherein the compound is 1-(6-chloro-2-pyridinyl)-4-piperidinamine, or salts, or solvates thereof.

22. A pharmaceutical composition comprising a therapeutically effective amount of at least one compound of claim 16 in admixture with at least one pharmaceutically acceptable carrier.

23. A method of neuroprotecting a patient comprising administering to a patient a therapeutically effective amount of at least one compound of claim 16.

24. The method of claim 23, wherein the patient has a disease selected from the group consisting of ischemic stroke, Alzheimer's disease, diabetic peripheral neuropathy, multiple sclerosis, amyotrophic lateral sclerosis, Huntington's disease and Parkinson's disease.
